(19) United States
(12) Patent Application Publication
(10) Pub. No.: US 2025/0240260 A1
(43) Pub. Date: Jul. 24, 2025

(54) METHOD, SYSTEM AND DEVICE FOR INTER-DEVICE COMMUNICATION, AND STORAGE MEDIUM

(71) Applicant: GOERTEK TECHNOLOGY CO., LTD., Qingdao (CN)

(72) Inventor: Jiancheng QI, Qingdao (CN)

(21) Appl. No.: 19/071,188

(22) Filed: Mar. 5, 2025

Related U.S. Application Data
(63) Continuation of application No. PCT/CN2023/126130, filed on Oct. 24, 2023.

(30) Foreign Application Priority Data
Oct. 26, 2022 (CN) 202211321248.9

(51) Int. Cl. H04L 51/214; H04L 51/23; H04W 84/20

(52) U.S. Cl. CPC H04L 51/214; H04L 51/23; H04W 84/20

(57) ABSTRACT

Disclosed is a method, a system and a device for inter-device communication, and a storage medium. The present application belongs to the technical field of communication. The first slave device of the present application uploads a to-be-processed data packet carrying health data to a mobile terminal. The mobile terminal determines whether the to-be-processed data packet needs to be processed. If not, the mobile terminal checks the destination address of the to-be-processed data packet and the Bluetooth address of the second slave device connected to local. If they are consistent, forwarding the to-be-processed data packet to the second slave device, to allow the second slave device display the health data to the user.

### CROSS-REFERENCE TO RELATED APPLICATIONS

The present application is a continuation application of International Application No. PCT/CN2023/126130, filed on Oct. 24, 2023, which claims priority to Chinese Patent Application No. 202211321248.9, filed on Oct. 26, 2022. The disclosures of the above-mentioned applications are incorporated herein by reference in their entireties.

### TECHNICAL FIELD

The present application relates to the technical field of communication, and in particular to a method for inter-device communication, a system for inter-device communication, a device for inter-device communication, and a storage medium.

### BACKGROUND

With the rise of the concept of the metaverse, more and more augmented reality (AR) products have entered people's daily lives. Generally, mobile terminals are connected to wearable devices and AR devices respectively by Bluetooth. As the main device, the mobile terminal establishes a Bluetooth connection with the AR device and a Bluetooth connection with the wearable device respectively. However, data cannot be shared between wearable devices and AR devices, that is, the user must use the mobile terminal to view the data uploaded by the wearable device or the AR device, which reduces the convenience of users viewing content.

### SUMMARY

The main purpose of the present application is to provide a method for inter-device communication, a system for inter-device communication, a device for inter-device communication, and a storage medium, aiming to solve the technical problem of reducing the convenience of users viewing content.

To achieve the above purpose, the present application provides a method for inter-device communication, applied to a mobile terminal, including:
in response to receiving a to-be-processed data packet, determining whether the to-be-processed data packet needs to be processed by analyzing a flag field in the to-be-processed data packet; the to-be-processed data packet being sent to local by a first slave device, and the to-be-processed data packet carries health data of a user collected by the first slave device;
in response to that the to-be-processed data packet does not need to be processed, obtaining a destination address of the to-be-processed data packet; and
in response to the destination address being consistent with a Bluetooth address of a second slave device connected to the local, forwarding the to-be-processed data packet to the second slave device to allow the user view the health data by the second slave device.

In a possible implementation of the present application, before the in response to receiving the to-be-processed data packet, determining whether the to-be-processed data packet needs to be processed by analyzing the flag field in the to-be-processed data packet, the method further includes:
sending the Bluetooth address of the second slave device paired successfully for a first time to the first slave device to allow the first slave device save the Bluetooth address of the second slave device, and sending a Bluetooth address of the first slave device paired successfully for a first time to the second slave device to allow the second slave device save the Bluetooth address of the first slave device.

In a possible implementation of the present application, before the in response to receiving the to-be-processed data packet, determining whether the to-be-processed data packet needs to be processed by analyzing the flag field in the to-be-processed data packet, the method further includes:
in response to receiving a heartbeat packet, determining whether the heartbeat packet needs to be forwarded; the heartbeat packet is sent from the first slave device to the local;
in response to that the heartbeat packet needs to be forwarded, sending the heartbeat packet to the second slave device;
in response to failing to receive a first response data packet fed back by the second slave device based on the heartbeat packet, feedbacking to the first slave device that the second slave device did not respond; and
in response to receiving the first response data packet fed back by the second slave device based on the heartbeat packet, forwarding the first response data packet to the first slave device to allow the first slave device determine that the second slave device being in a connected state with the local.

In a possible implementation of the present application, after the in response to receiving the to-be-processed data packet, determining whether the to-be-processed data packet needs to be processed by analyzing the flag field in the to-be-processed data packet, the method further includes:
in response to the to-be-processed data packet needing to be processed, extracting the health data of the user carried in the to-be-processed data packet and saving to the local.

The present application further provides a method for inter-device communication, applied to a first slave device, including:
determining whether a mobile terminal is disconnected from local; and
in response to the mobile terminal being not disconnected from the local, encapsulating collected user's health data into a to-be-processed data packet, and sending the to-be-processed data packet to the mobile terminal to allow the mobile terminal to process the to-be-processed data packet; destination address of the to-be-processed data packet is Bluetooth address of a second slave device or Bluetooth address of the mobile terminal.

In a possible implementation of the present application, after the determining whether the mobile terminal is disconnected from the local, the method further includes:
in response to the mobile terminal being disconnected from the local, obtaining the Bluetooth address of the second slave device by querying a local Bluetooth address book; the Bluetooth address of the second slave device being sent to the local after the mobile terminal completes a first pairing with the second slave device;
initiating a connection request to the second slave device based on the Bluetooth address of the second slave device; and
in response to connecting successfully, sending the to-be-processed data packet to the second slave device to allow the second slave device display the health data to the user.

In a possible implementation of the present application, the determining whether the mobile terminal is disconnected from the local includes:
sending a heartbeat packet to the mobile terminal; destination address of the heartbeat packet is Bluetooth address of the mobile terminal or the Bluetooth address of the second slave device; and
in response to receiving feedback information that the second slave device does not respond within a consecutive preset number of times, and failing to receive a second response data packet fed back by the mobile terminal based on the heartbeat packet, determining that the mobile terminal is disconnected from the local; the feedback information is sent by the mobile terminal to the local.

The present application further provides a system for inter-device communication, including: a mobile terminal, a first slave device and a second slave device;
the mobile terminal is configured for determining whether the to-be-processed data packet needs to be processed by analyzing a flag field in the to-be-processed data packet in response to receiving a to-be-processed data packet, the to-be-processed data packet being sent to local by the first slave device, and the to-be-processed data packet carries health data of a user collected by the first slave device; the mobile terminal is further configured for obtaining a destination address of the to-be-processed data packet in response to that the to-be-processed data packet does not need to be processed; the mobile terminal is further configured for forwarding the to-be-processed data packet to the second slave device to allow the user view the health data by the second slave device in response to the destination address being consistent with a Bluetooth address of the second slave device connected to the local; and
the first slave device is configured for determining whether the mobile terminal is disconnected from the local; and the first slave device is further configured for encapsulating collected user's health data into the to-be-processed data packet, and sending the to-be-processed data packet to the mobile terminal in response to the mobile terminal being not disconnected from the local, so as to allow the mobile terminal to process the to-be-processed data packet; destination address of the to-be-processed data packet is the Bluetooth address of the second slave device or Bluetooth address of the mobile terminal.

The present application further provides a device for inter-device communication, including: a memory, a processor, and a program for inter-device communication stored in the memory and executable on the processor, the program for inter-device communication is configured for implementing the method for inter-device communication as described in any one of the above.

The present application further provides a storage medium, a program for inter-device communication is stored on the storage medium, and the method for inter-device communication as described in any one of the above are implemented when the program for inter-device communication is executed by a processor.

The present application provides a method for inter-device communication. Compared with the prior art, a user must use a mobile terminal to view data uploaded by a wearable device or an AR device, which reduces the convenience of the user viewing content. In the present application, a first slave device uploads a to-be-processed data packet carrying health data to a mobile terminal. The mobile terminal determines whether the to-be-processed data packet needs to be processed. If the to-be-processed data packet does not need to be processed, the mobile terminal checks the destination address of the to-be-processed data packet and the Bluetooth address of a second slave device connected to local. If they are consistent, forwarding the to-be-processed data packet to the second slave device, so that the second slave device can display the health data to the user, thereby realizing communication between the first slave device and the second slave device. The user can directly view the health data by the second slave device, rather than only being able to view health data by mobile terminals. When the first slave device is a wearable device, the second slave device is an AR device. When the first slave device is the AR device, the second slave device is the wearable device. That is, the present application realizes the communication between the wearable device and the AR device, and improves the convenience of users viewing content.

### BRIEF DESCRIPTION OF THE DRAWINGS

FIG. 1 is a flowchart of a method for inter-device communication according to an embodiment the present application.

FIG. 2 is a first scenario diagram of the method for inter-device communication according to an embodiment of the present application.

FIG. 3 is a flowchart of a method for inter-device communication according to another embodiment the present application.

FIG. 4 is a flowchart of a method for inter-device communication according to another embodiment the present application.

FIG. 5 is a flowchart of a method for inter-device communication according to another embodiment the present application.

FIG. 6 is a flowchart of a method for inter-device communication according to another embodiment the present application.

FIG. 7 is a flowchart of a method for inter-device communication according to another embodiment the present application.

FIG. 8 is a flowchart of a method for inter-device communication according to another embodiment the present application.

FIG. 9 is a second scenario diagram of the method for inter-device communication according to an embodiment of the present application.

FIG. 10 is a third scenario diagram of the method for inter-device communication according to an embodiment of the present application.

FIG. 11 is a structural schematic view of a device for inter-device communication of a hardware operating environment involved in an embodiment of the present application.

The purpose, features and advantages of the present application will be further described in conjunction with the embodiments and with reference to the accompanying drawings.

### DETAILED DESCRIPTION OF THE EMBODIMENTS

It should be understood that the specific embodiments described herein are only used to explain the present application and are not used to limit the present application. Although the terms “first”, “second”, “third”, etc. may be used in the present application to describe various information, these information should not be limited to these terms. These terms are only used to distinguish the same type of information from each other. For example, without departing from the scope of the present application, the first information may also be referred to as the second information, and similarly, the second information may also be referred to as the first information.

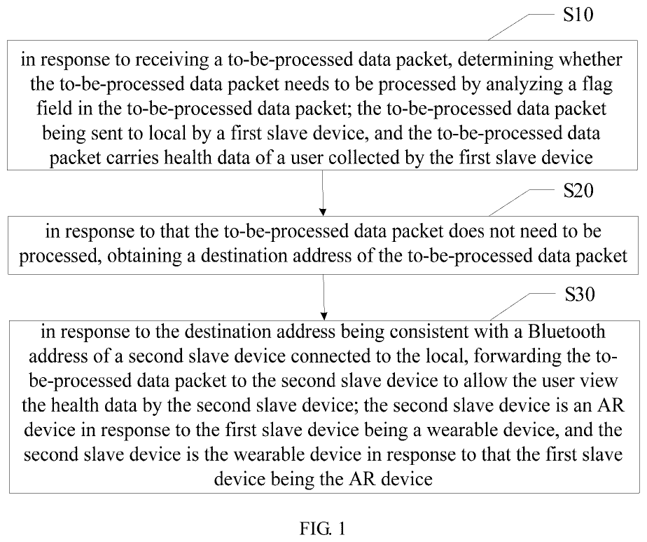

The embodiment of the present application provides a method for inter-device communication. Referring to FIG. 1, in this embodiment, the method for inter-device communication is applied to a mobile terminal, and the method for inter-device communication includes:

Step S10: in response to receiving a to-be-processed data packet, determining whether the to-be-processed data packet needs to be processed by analyzing a flag field in the to-be-processed data packet; the to-be-processed data packet being sent to local by a first slave device, and the to-be-processed data packet carries health data of a user collected by the first slave device.

Step S20: in response to that the to-be-processed data packet does not need to be processed, obtaining a destination address of the to-be-processed data packet.

Step S30: in response to the destination address being consistent with a Bluetooth address of a second slave device connected to the local, forwarding the to-be-processed data packet to the second slave device to allow the user view the health data by the second slave device; the second slave device is an augmented reality (AR) device in response to the first slave device being a wearable device, and the second slave device is the wearable device in response to that the first slave device being the AR device.

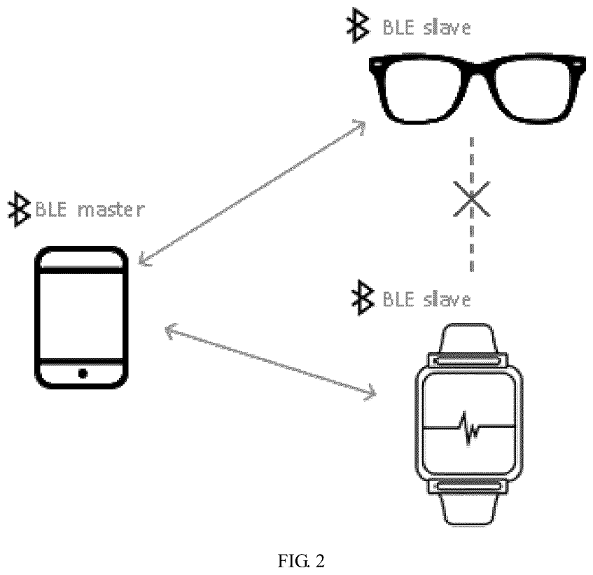

Research and development background of this embodiment: as shown in FIG. 2, the mobile terminal is connected to the wearable device and the AR device respectively by Bluetooth Low Energy (BLE). The mobile terminal acts as the master device (BLE master), and is connected to the AR device (the first slave device, BLE slave) by BLE, and is connected to the wearable device (the second slave device, BLE slave) by BLE. However, the wearable device and the AR device cannot be connected by BLE communication, that is, data cannot be shared between the wearable device and the AR device (between two slave devices).

Specifically, the mobile terminal, the wearable device, and the AR device only support the BLE Bluetooth protocol, and do not support the traditional Bluetooth protocol (Bluetooth, BT).

Specifically, a BLE slave device cannot simultaneously serve as a BLE master device and connect to other BLE slave devices via BLE communication, that is, an AR device serving as a BLE slave device cannot simultaneously connect to a wearable device via BLE communication.

This embodiment aims to: use a mobile terminal as a data transfer station, share the health data of the user collected by the wearable device with the AR device, and solve the problem that data cannot be shared between the wearable device and the AR device.

In this embodiment, a method for inter-device communication is applied to a mobile terminal in a system for inter-device communication, and the system for inter-device communication further includes a wearable device and an AR device, at least one wearable device and at least one AR device are provided.

As an example, the mobile terminal can be a mobile phone, a notebook, a tablet computer, etc., without specific limitation.

As an example, the wearable device may be a portable device such as a watch, a wristband, a ring, an armband, etc., without specific limitation.

As an example, the AR device may be AR glasses and AR helmets, etc., without specific limitation.

As an example, the wearable device is configured to collect the user's health data, and further to display the content shared by the AR device to the user. The AR device is configured to collect the user's health data, and further to display the content shared by the wearable device to the user.

As an example, the user's health data may be heart rate, blood oxygen and other vital signs status data, and further may be step number, exercise mileage and other exercise data, etc., without specific limitation.

As an example, the to-be-processed data packet includes a flag field, a destination address field (Bluetooth Mac address, Bluetooth address), and payload (payload data), and the payload data is the health data of the user collected by the first slave device. Referring to FIG. 10, the flag field can occupy 0 bit to 8 bits, the Bluetooth Mac address occupies 0 bit to 64 bits, and the payload data can occupy 0 bit to 224 bits.

The specific steps are as follows.

Step S10: in response to receiving a to-be-processed data packet, determining whether the to-be-processed data packet needs to be processed by analyzing a flag field in the to-be-processed data packet; the to-be-processed data packet being sent to local by a first slave device, and the to-be-processed data packet carries health data of a user collected by the first slave device.

As an example, the first slave device uploads the to-be-processed data packet carrying the health data to the mobile terminal, and the mobile terminal determines whether the to-be-processed data packet needs to be processed.

Specifically, the mobile terminal determines whether the to-be-processed data packet needs to be processed by analyzing the flag field in the to-be-processed data packet.

As an example, the flag field can be set to 0 or 1, or can be set to as A, B or other flag. If the flag field is 0 or A, determining that the to-be-processed data needs to be processed. If the flag field is 1 or B, determining that the to-be-processed data does not need to be processed.

As an example, when the to-be-processed data needs to be processed, the processing method of the to-be-processed data may be to local storage, analysis, etc., and the specific method is not limited.

As an example, when the to-be-processed data does not need to be processed, the processing method of the to-be-processed data may be to forward, discard, etc. from local, and the specific method is not limited.

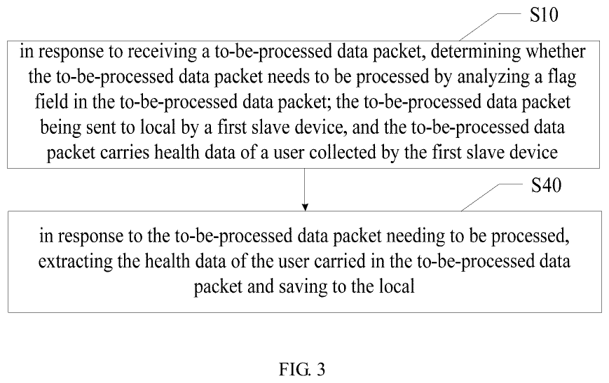

In this embodiment, after the in response to receiving the to-be-processed data packet, determining whether the to-be-processed data packet needs to be processed by analyzing the flag field in the to-be-processed data packet, as shown in FIG. 3, the method further includes:

Step S40, in response to the to-be-processed data packet needing to be processed, extracting the health data of the user carried in the to-be-processed data packet and saving to the local.

As an example, if the flag field is 0 or A, determining that the to-be-processed data needs to be processed, and extracting the health data of the user carried in the to-be-processed data packet.

As an example, it may be that extracting the payload field in the to-be-processed data packet and obtaining the payload data, that is, obtaining the health data of the user, saving the health data of the user locally, and updating the local original data to allow the user view the health data by the mobile terminal.

Step S20: in response to that the to-be-processed data packet does not need to be processed, obtaining a destination address of the to-be-processed data packet.

As an example, if the flag field is 1 or B, determining that the to-be-processed data packet does not need to be processed, and obtaining the destination address of the to-be-processed data packet.

Specifically, it may be that by analyzing the destination address field of the to-be-processed data packet, obtaining the destination address of the to-be-processed data packet; the destination address is a Bluetooth address, and the destination address can be the Bluetooth address of the second slave device and the Bluetooth address of the first slave device.

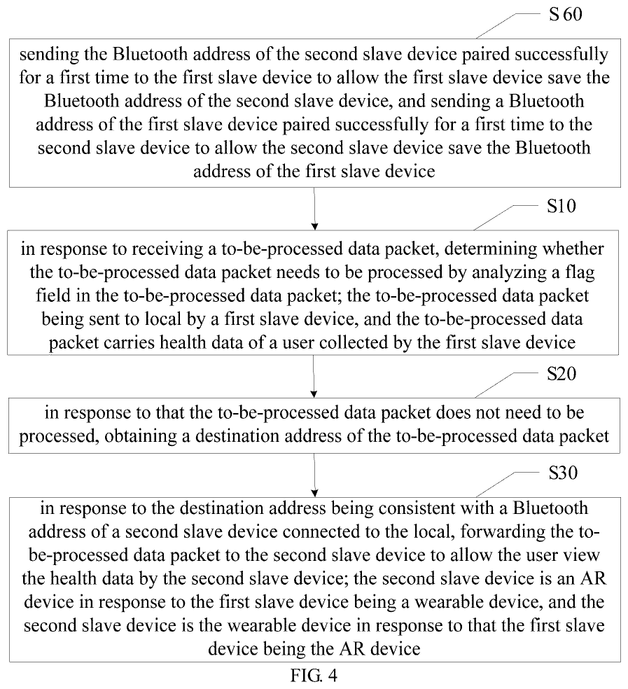

In this embodiment, before the in response to receiving the to-be-processed data packet, determining whether the to-be-processed data packet needs to be processed by analyzing the flag field in the to-be-processed data packet, as shown in FIG. 4, the method further includes:

Step S60: sending the Bluetooth address of the second slave device paired successfully for a first time to the first slave device to allow the first slave device save the Bluetooth address of the second slave device, and sending a Bluetooth address of the first slave device paired successfully for a first time to the second slave device to allow the second slave device save the Bluetooth address of the first slave device.

As an example, the mobile terminal sends the Bluetooth address of the second slave device that is paired successfully for the first time to the first slave device, and the first slave device saves the Bluetooth address of the second slave device to the local Bluetooth address book, and the local Bluetooth address book of the first slave device stores the Bluetooth address of the mobile terminal and the Bluetooth address of the second slave device; the mobile terminal sends the Bluetooth address of the first slave device that is paired successfully for the first time to the second slave device, the second slave device saves the Bluetooth address of the first slave device to the local Bluetooth address book, and the local Bluetooth address book of the second slave device stores the Bluetooth address of the mobile terminal and the Bluetooth address of the first slave device.

As an example, after the first slave device collects the user's health data, the first slave device can choose to send the user's health data to the mobile terminal or to the second slave device. Therefore, by querying the local Bluetooth address book, based on the Bluetooth address in the Bluetooth address book, the first slave device sends the user's health data to the mobile terminal or to the second slave device.

In this embodiment, as shown in FIG. 5, before the in response to receiving the to-be-processed data packet, determining whether the to-be-processed data packet needs to be processed by analyzing the flag field in the to-be-processed data packet, the method further includes:

Step S70, in response to receiving a heartbeat packet, determining whether the heartbeat packet needs to be forwarded; the heartbeat packet is sent from the first slave device to the local.

In this embodiment, before the first slave device sends the to-be-processed data packet to the mobile terminal, it is necessary to send the heartbeat packet to the mobile terminal to determine whether to be disconnected from the mobile terminal. However, the destination address of the heartbeat packet is the Bluetooth address of the mobile terminal or the Bluetooth address of the second slave device. That is, when the mobile terminal receives the heartbeat packet, it is necessary to determine whether the heartbeat packet needs to be forwarded by analyzing the flag field of the heartbeat packet.

Step S80: in response to that the heartbeat packet needs to be forwarded, sending the heartbeat packet to the second slave device.

As an example, if the flag field is 1 or B, determining that the heartbeat packet needs to be forwarded, and the mobile terminal forwards the heartbeat packet to the second slave device.

Step S90: in response to failing to receive a first response data packet fed back by the second slave device based on the heartbeat packet, feedbacking to the first slave device that the second slave device did not respond.

As an example, if the mobile terminal does not receive the first response data packet fed back by the second slave device based on the heartbeat packet, then determining that the second slave device being in a disconnected state with the mobile terminal, and the mobile terminal sends the feedback information that the second slave device did not respond to the first slave device, to allow the first slave device know that the second slave device is disconnected from the mobile terminal, so as to avoid misjudging the disconnection between the mobile terminal and the first slave device.

Step S100, in response to receiving the first response data packet fed back by the second slave device based on the heartbeat packet, forwarding the first response data packet to the first slave device to allow the first slave device determine that the second slave device being in a connected state with the local.

As an example, if the mobile terminal receives the first response data packet fed back by the second slave device based on the heartbeat packet, the mobile terminal forwards the first response data packet to the first slave device, to allow the first slave device determine that the second slave device is in a connected state with the local.

Step S30: in response to the destination address being consistent with a Bluetooth address of a second slave device connected to the local, forwarding the to-be-processed data packet to the second slave device to allow the user view the health data by the second slave device.

As an example, the scenario targeted by this embodiment is that the mobile terminal is in a connected state with the second slave device, and at the same time, the mobile terminal is in a connected state with the first slave device.

Specifically, when the mobile terminal determines that the to-be-processed data packet does not need to be processed, it is necessary to forward the to-be-processed data packet to the second slave device connected to the local.

Specifically, based on the obtained destination address of the to-be-processed data packet, determining whether the destination address is consistent with the Bluetooth address of the second slave device connected to the local.

As an example, if the destination address is inconsistent with the Bluetooth address of the second slave device connected to the local, prompting the user that the second slave device has been disconnected and cannot share data, to allow the user connect the second slave device to the mobile terminal in time.

As an example, if the destination address is consistent with the Bluetooth address of the second slave device connected to the local, extracting the health data of the user carried in the to-be-processed data packet, repackaging and forwarding the health data of the user carried in the to-be-processed data packet to the second slave device, to allow the second slave device display the health data to the user.

In this embodiment, the first slave device uploads a to-be-processed data packet carrying health data to the mobile terminal, and the mobile terminal determines whether the to-be-processed data packet needs to be processed. If the to-be-processed data packet does not need to be processed, the mobile terminal checks the destination address of the to-be-processed data packet and the Bluetooth address of the second slave device connected to the local. If they are consistent, forwarding the to-be-processed data packet to the second slave device, and the user can view the health data by the second slave device. If the to-be-processed data packet needs to be processed, the mobile terminal extracts the health data of the user carried in the to-be-processed data packet and saves to local, so as to realize communication among the wearable device, the mobile terminal and the AR device. The user can view the health data by the wearable device or the AR device, and can further view the health data by the mobile terminal, rather than only being able to view health data by mobile terminals, thus improving the convenience of users viewing content.

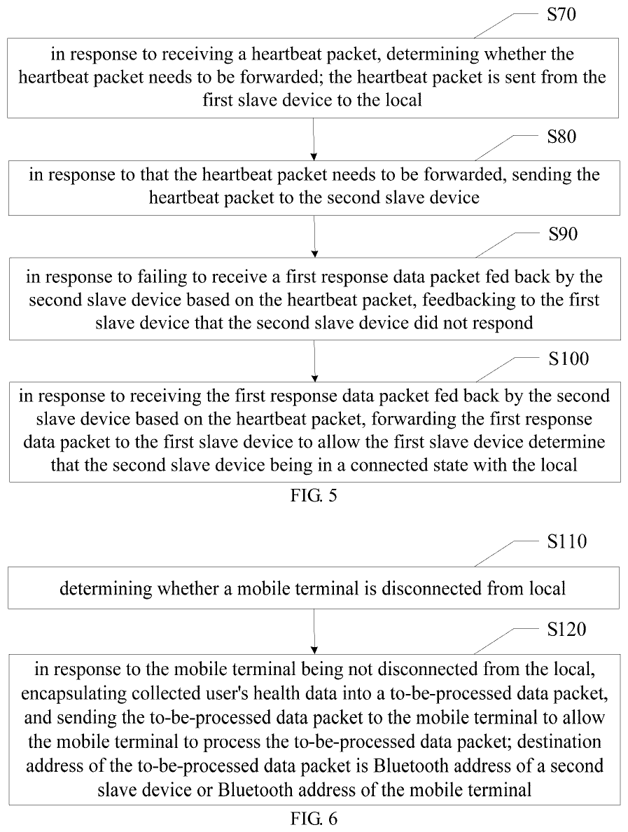

Further, as shown in FIG. 6, based on the first embodiment of the present application, another embodiment of the present application is provided. In this embodiment, the step of inter-device communication applied to the first slave device includes:

Step S110, determining whether a mobile terminal is disconnected from local.

In this embodiment, the method for inter-device communication is applied to the first slave device in the system for inter-device communication, and the system for inter-device communication further includes a mobile terminal and a second slave device. When the first slave device is a wearable device, the second slave device is an AR device. When the first slave device is the AR device, the second slave device is the wearable device.

In this embodiment, before the first slave device sends the to-be-processed data packet to the mobile terminal, it is necessary to first determine whether the mobile terminal is disconnected from the local area. If it is not disconnected, the first slave device can directly send the to-be-processed data packet to the mobile terminal to achieve communication between the first slave device and the mobile terminal and communication between the first slave device the second slave device (refer to the first embodiment). If it is disconnected, the first slave device needs to be directly connected to the second slave device to achieve communication.

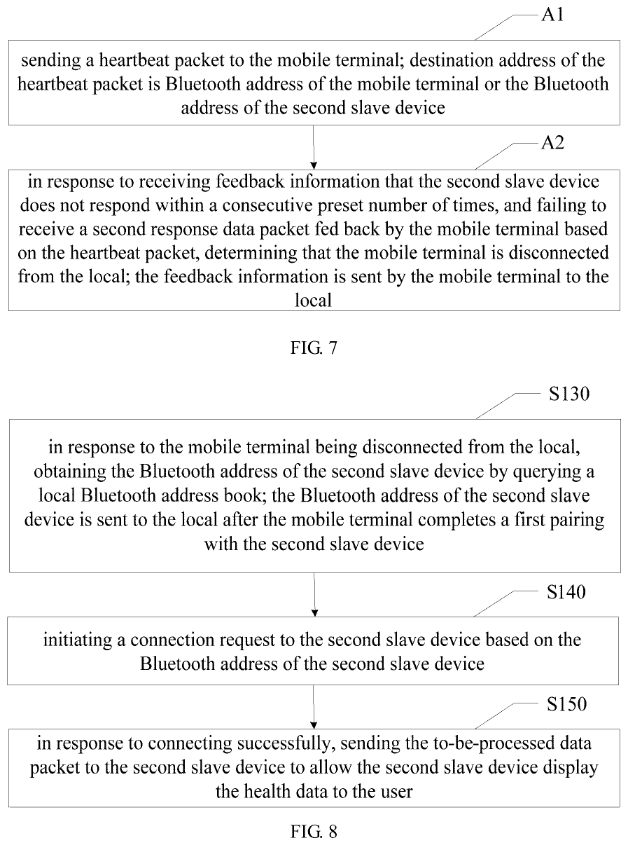

In this embodiment, as shown in FIG. 7, the determining whether the mobile terminal is disconnected from the local includes:

Step A1, sending a heartbeat packet to the mobile terminal; destination address of the heartbeat packet is Bluetooth address of the mobile terminal or the Bluetooth address of the second slave device.

In this embodiment, the method of determining whether the mobile terminal is disconnected from the local is: the first slave device sends a heartbeat packet to the mobile terminal; if the first slave device needs to determine whether the local is in a connection status with the mobile terminal, the destination address of the heartbeat packet sent is the Bluetooth address of the mobile terminal; if the first slave device needs to determine whether the second slave device is in a connection status with the mobile terminal, the destination address of the heartbeat packet sent is the Bluetooth address of the mobile terminal.

Step A2: in response to receiving feedback information that the second slave device does not respond within a consecutive preset number of times, and failing to receive a second response data packet fed back by the mobile terminal based on the heartbeat packet, determining that the mobile terminal is disconnected from the local; the feedback information is sent by the mobile terminal to the local.

As an example, if the first slave device receives the feedback information that the second slave device does not respond sent by the mobile terminal to the local within a consecutive preset number of times, determining that the second slave device is disconnected from the mobile terminal. If the second response data packet fed back by the mobile terminal based on the heartbeat packet is not received within the consecutive preset number of times, determining that the mobile terminal is disconnected from the first slave device. At this time, the wearable device, the mobile terminal and the AR device are not connected to each other.

Otherwise, determining that the mobile terminal is not disconnected from the first slave device, that is, the first slave device cannot be directly connected to the second slave device by the BLE.

Step S120: in response to the mobile terminal being not disconnected from the local, encapsulating collected user's health data into a to-be-processed data packet, and sending the to-be-processed data packet to the mobile terminal to allow the mobile terminal to process the to-be-processed data packet; destination address of the to-be-processed data packet is Bluetooth address of a second slave device or Bluetooth address of the mobile terminal.

In this embodiment, if determining that the connection is not disconnected, the first slave device can only share the user's health data to the second slave device by the mobile terminal. Specifically, the first slave device encapsulates the collected user's health data into a to-be-processed data packet, and sends the to-be-processed data packet to the mobile terminal, to allow the mobile terminal process the to-be-processed data packet (refer to the first embodiment).

In this embodiment, as shown in FIG. 8, after the determining whether the mobile terminal is disconnected from the local, the method further includes:

Step S130, in response to the mobile terminal being disconnected from the local, obtaining the Bluetooth address of the second slave device by querying a local Bluetooth address book; the Bluetooth address of the second slave device is sent to the local after the mobile terminal completes a first pairing with the second slave device.

As an example, if the first slave device determines that the mobile terminal is disconnected from the first slave device, querying the local Bluetooth address book, and the local Bluetooth address book stores the Bluetooth address of the second slave device and the Bluetooth address of the mobile terminal.

Specifically, obtaining the Bluetooth address of the second slave device by querying the local Bluetooth address book.

Step S140, initiating a connection request to the second slave device based on the Bluetooth address of the second slave device.

As an example, when the first slave device encapsulates the health data of the user, modifying the destination address field to the Bluetooth address of the second slave device, and the first slave device sends the re-encapsulated data packet to the second slave device, and initiates a connection request to the second slave device.

Step S150, in response to connecting successfully, sending the to-be-processed data packet to the second slave device to allow the second slave device display the health data to the user.

As an example, if the connection is successful, the first slave device can directly encapsulate the collected user's health data into a to-be-processed data packet, and send the to-be-processed data packet to the second slave device, to allow the second slave device display the health data to the user.

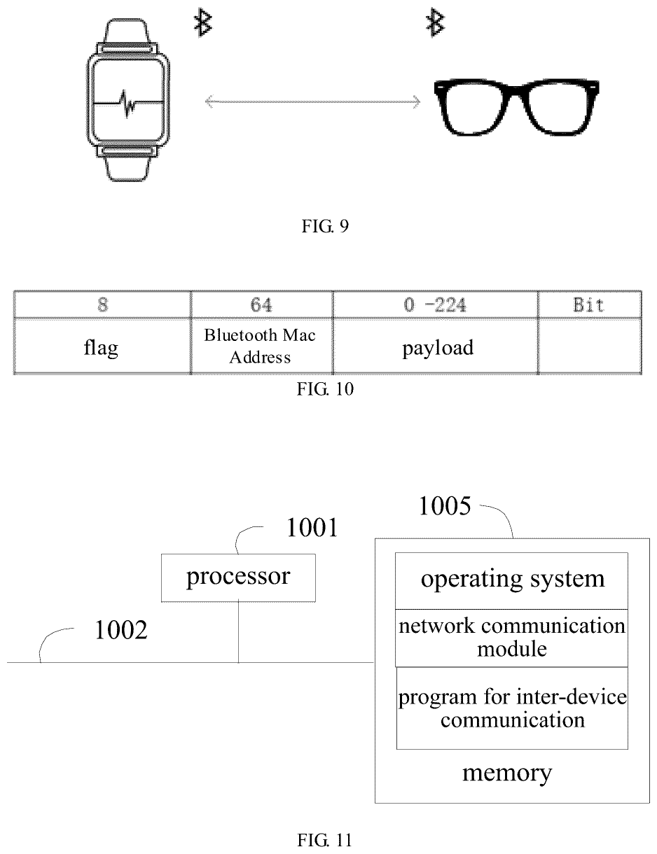

In this embodiment, if the mobile terminal is lost or not around, the user cannot view the health data by the mobile terminal. Referring to FIG. 9, at this time, the first slave device can, when determining that the mobile terminal is disconnected from the local, initiate a connection request to the second slave device based on the Bluetooth address of the second slave device. If the connection is successful, the to-be-processed data packet can be directly sent to the second slave device, to allow the second slave device display the health data to the user. That is, when the mobile terminal is not around, a connection is established between the AR device and the wearable device, the user can directly view the health data by the AR device or the wearable device, further improving the convenience of the user viewing the content.

Referring to FIG. 11, FIG. 11 is a schematic diagram of the device structure of the hardware operating environment involved in the embodiment of the present application.

As shown in FIG. 11, the device for inter-device communication may include a processor 1001, a memory 1005, and a communication bus 1002. The communication bus 1002 is configured to realize the connection and communication between the processor 1001 and the memory 1005.

In an embodiment, the device for inter-device communication may further include a user interface, a network interface, a camera, a Radio Frequency (RF) circuit, a sensor, a Wireless Fidelity (WiFi) module, and the like. The user interface may include a display, an input submodule such as a keyboard. The optional user interface may further include a standard wired interface and a wireless interface. The network interface may include a standard wired interface and a wireless interface (such as a WI-FI interface).

Those skilled in the art can understand that the device structure for inter-device communication shown in FIG. 11 does not constitute a limitation on the device for inter-device communication, and may include more or fewer components than shown in the figure, or combine certain components, or arrange components differently.

As shown in FIG. 11, the memory 1005 as a storage medium may include an operating system, a network communication module, and a program for inter-device communication. The operating system is a program that manages and controls the hardware and software resources of the device for inter-device communication, and supports the operation of the program for inter-device communication and other software and/or programs. The network communication module is configured to realize the communication between the components inside the memory 1005, and the communication with other hardware and software in the system for inter-device communication.

In the device for inter-device communication shown in FIG. 11, the processor 1001 is configured to execute the program for inter-device communication stored in the memory 1005, and realize the method for inter-device communication described in any of the above items.

The specific implementation methods of the device for inter-device communication in the present application are basically consistent with the various embodiments of the method for inter-device communication described above, and will not be repeated here.

The present application further provides a system for inter-device communication, the system includes a mobile terminal, a first slave device and a second slave device.

The mobile terminal is configured for determining whether the to-be-processed data packet needs to be processed by analyzing a flag field in the to-be-processed data packet in response to receiving a to-be-processed data packet, the to-be-processed data packet being sent to local by the first slave device, and the to-be-processed data packet carries health data of a user collected by the first slave device. The mobile terminal is further configured for obtaining a destination address of the to-be-processed data packet in response to that the to-be-processed data packet does not need to be processed. The mobile terminal is further configured for forwarding the to-be-processed data packet to the second slave device to allow the user view the health data by the second slave device in response to the destination address being consistent with a Bluetooth address of the second slave device connected to the local

In a possible implementation, the mobile terminal is further configured for sending the Bluetooth address of the second slave device paired successfully for a first time to the first slave device to allow the first slave device save the Bluetooth address of the second slave device, and sending a Bluetooth address of the first slave device paired successfully for a first time to the second slave device to allow the second slave device save the Bluetooth address of the first slave device.

In a possible implementation, the mobile terminal is further configured for determining whether the heartbeat packet needs to be forwarded in response to receiving a heartbeat packet; the heartbeat packet is sent from the first slave device to the local. The mobile terminal is further configured for sending the heartbeat packet to the second slave device in response to that the heartbeat packet needs to be forwarded. The mobile terminal is further configured for feedbacking to the first slave device that the second slave device did not respond in response to failing to receive a first response data packet fed back by the second slave device based on the heartbeat packet. The mobile terminal is further configured for forwarding the first response data packet to the first slave device in response to receiving the first response data packet fed back by the second slave device based on the heartbeat packet, so as to allow the first slave device determine that the second slave device being in a connected state with the local.

In a possible implementation, the mobile terminal is further configured for extracting the health data of the user carried in the to-be-processed data packet and saving to the local in response to the to-be-processed data packet needing to be processed.

The first slave device is configured for determining whether the mobile terminal is disconnected from the local. The first slave device is further configured for encapsulating collected user's health data into the to-be-processed data packet, and sending the to-be-processed data packet to the mobile terminal in response to the mobile terminal being not disconnected from the local, so as to allow the mobile terminal to process the to-be-processed data packet; destination address of the to-be-processed data packet is the Bluetooth address of the second slave device or Bluetooth address of the mobile terminal.

In a possible implementation, the first slave device is further configured for obtaining the Bluetooth address of the second slave device by querying a local Bluetooth address book in response to the mobile terminal being disconnected from the local. The Bluetooth address of the second slave device being sent to the local after the mobile terminal completes a first pairing with the second slave device. The first slave device is further configured for initiating a connection request to the second slave device based on the Bluetooth address of the second slave device. The first slave device is further configured for sending the to-be-processed data packet to the second slave device in response to connecting successfully, so as to allow the second slave device display the health data to the user.

In a possible implementation, the first slave device is further configured for sending a heartbeat packet to the mobile terminal. The destination address of the heartbeat packet is Bluetooth address of the mobile terminal or the Bluetooth address of the second slave device. The first slave device is further configured for determining that the mobile terminal is disconnected from the local in response to receiving feedback information that the second slave device does not respond within a consecutive preset number of times and failing to receive a second response data packet fed back by the mobile terminal based on the heartbeat packet. The feedback information is sent by the mobile terminal to the local.

The specific implementation of the system for inter-device communication of the present application is basically the same as the various embodiments of the method for inter-device communication described above, and will not be repeated here.

The present application further provides a storage medium, a program for inter-device communication is stored on the storage medium, and the method for inter-device communication in any one of the above are implemented when the program for inter-device communication is executed by a processor.

The specific implementation method of the storage medium of the present application is basically the same as the various embodiments of the inter-device communication described above, and will not be repeated here.

It should be noted that, in the present application, the terms “include”, “comprise” or any other variants thereof are intended to cover non-exclusive inclusion, so that a process, method, article or system including a series of elements includes not only those elements, but also further includes other elements not explicitly listed, or further includes elements inherent to such process, method, article or system. In the absence of further restrictions, an element defined by the sentence “includes a . . . ” does not exclude the existence of other identical elements in the process, method, article or system including the element.

The serial numbers of the embodiments of the present application are only for description and do not represent the advantages and disadvantages of the embodiments.

By the description of the above implementation methods, those skilled in the art can clearly understand that the above embodiments can be implemented by means of software plus the necessary general hardware platform; certainly, the above embodiments can also be implemented by hardware, but in many cases the former is a better implementation method. Based on this understanding, the technical solution of the present application can be essentially or partly contributed to the prior art in the form of a software product, which is stored in a storage medium (such as ROM/RAM (Random-Access Memory/Read-Only Memory), disk, CD (Compact Disc)) as described above, including several instructions for enabling a terminal device (which can be a mobile phone, computer, server, air conditioner, or network device, etc.) to execute the methods described in each embodiment of the present application.

The above are only some embodiments of the present application, and do not limit the scope of the present application. Any equivalent structure or equivalent process transformation made by using the contents of the specification and drawings of the present application, or directly or indirectly applied in other related technical fields, are also included in the scope of the present application.

What is claimed is:

1. A method for inter-device communication, applied to a mobile terminal, comprising:
in response to receiving a to-be-processed data packet, determining whether the to-be-processed data packet needs to be processed by analyzing a flag field in the to-be-processed data packet; wherein the to-be-processed data packet being sent to local by a first slave device, and the to-be-processed data packet carries health data of a user collected by the first slave device;
in response to that the to-be-processed data packet does not need to be processed, obtaining a destination address of the to-be-processed data packet; and
in response to the destination address being consistent with a Bluetooth address of a second slave device connected to the local, forwarding the to-be-processed data packet to the second slave device to allow the user view the health data by the second slave device; wherein the second slave device is an augmented reality (AR) device in response to the first slave device being a wearable device, and the second slave device is the wearable device in response to that the first slave device being the AR device.

2. The method for inter-device communication according to claim 1, wherein before the in response to receiving the to-be-processed data packet, determining whether the to-be-processed data packet needs to be processed by analyzing the flag field in the to-be-processed data packet, the method further comprises:
sending the Bluetooth address of the second slave device paired successfully for a first time to the first slave device to allow the first slave device save the Bluetooth address of the second slave device, and sending a Bluetooth address of the first slave device paired successfully for a first time to the second slave device to allow the second slave device save the Bluetooth address of the first slave device.

3. The method for inter-device communication according to claim 1, wherein before the in response to receiving the to-be-processed data packet, determining whether the to-be-processed data packet needs to be processed by analyzing the flag field in the to-be-processed data packet, the method further comprises:
in response to receiving a heartbeat packet, determining whether the heartbeat packet needs to be forwarded; wherein the heartbeat packet is sent from the first slave device to the local;
in response to that the heartbeat packet needs to be forwarded, sending the heartbeat packet to the second slave device;
in response to failing to receive a first response data packet fed back by the second slave device based on the heartbeat packet, feedbacking to the first slave device that the second slave device did not respond; and
in response to receiving the first response data packet fed back by the second slave device based on the heartbeat packet, forwarding the first response data packet to the first slave device to allow the first slave device determine that the second slave device being in a connected state with the local.

4. The method for inter-device communication according to claim 2, wherein after the in response to receiving the to-be-processed data packet, determining whether the to-be-processed data packet needs to be processed by analyzing the flag field in the to-be-processed data packet, the method further comprises:
in response to the to-be-processed data packet needing to be processed, extracting the health data of the user carried in the to-be-processed data packet and saving to the local.

5. A method for inter-device communication, applied to a first slave device, comprising:
determining whether a mobile terminal is disconnected from local; and
in response to the mobile terminal being not disconnected from the local, encapsulating collected user's health data into a to-be-processed data packet, and sending the to-be-processed data packet to the mobile terminal to allow the mobile terminal to process the to-be-processed data packet; wherein destination address of the to-be-processed data packet is Bluetooth address of a second slave device or Bluetooth address of the mobile terminal.

6. The method for inter-device communication according to claim 1, wherein after the determining whether the mobile terminal is disconnected from the local, the method further comprises:
in response to the mobile terminal being disconnected from the local, obtaining the Bluetooth address of the second slave device by querying a local Bluetooth address book; wherein the Bluetooth address of the second slave device being sent to the local after the mobile terminal completes a first pairing with the second slave device;
initiating a connection request to the second slave device based on the Bluetooth address of the second slave device; and
in response to connecting successfully, sending the to-be-processed data packet to the second slave device to allow the second slave device display the health data to the user.

7. The method for inter-device communication according to claim 1, wherein the determining whether the mobile terminal is disconnected from the local comprises:
sending a heartbeat packet to the mobile terminal; wherein destination address of the heartbeat packet is Bluetooth address of the mobile terminal or the Bluetooth address of the second slave device; and
in response to receiving feedback information that the second slave device does not respond within a consecutive preset number of times, and failing to receive a second response data packet fed back by the mobile terminal based on the heartbeat packet, determining that the mobile terminal is disconnected from the local; wherein the feedback information is sent by the mobile terminal to the local.

8. A system for inter-device communication, comprising: a mobile terminal, a first slave device and a second slave device;
wherein the mobile terminal is configured for determining whether the to-be-processed data packet needs to be processed by analyzing a flag field in the to-be-processed data packet in response to receiving a to-be-processed data packet, wherein the to-be-processed data packet being sent to local by the first slave device, and the to-be-processed data packet carries health data of a user collected by the first slave device; the mobile terminal is further configured for obtaining a destination address of the to-be-processed data packet in response to that the to-be-processed data packet does not need to be processed; the mobile terminal is further configured for forwarding the to-be-processed data packet to the second slave device to allow the user view the health data by the second slave device in response to the destination address being consistent with a Bluetooth address of the second slave device connected to the local; and
the first slave device is configured for determining whether the mobile terminal is disconnected from the local; and the first slave device is further configured for encapsulating collected user's health data into the to-be-processed data packet, and sending the to-be-processed data packet to the mobile terminal in response to the mobile terminal being not disconnected from the local, so as to allow the mobile terminal to process the to-be-processed data packet; wherein destination address of the to-be-processed data packet is the Bluetooth address of the second slave device or Bluetooth address of the mobile terminal.

9. A device for inter-device communication, comprising: a memory, a processor, and a program for inter-device communication stored in the memory and executable on the processor, wherein the program for inter-device communication is configured for implementing the method for inter-device communication according to claim 1.

10. A non-transitory computer-readable storage medium, wherein a program for inter-device communication is stored on the non-transitory computer-readable storage medium, and the method for inter-device communication according to claim 1 are implemented when the program for inter-device communication is executed by a processor.
